(19) United States
(12) Patent Application Publication
(10) Pub. No.: US 2022/0176064 A1
(43) Pub. Date: Jun. 9, 2022

(54) THIN-PROFILE DELIVERY APPARATUS AND MEDIA CARTRIDGES

(71) Applicant: SENSORY CLOUD, LLC, Boston, MA (US)

(72) Inventors: David A. Edwards, Boston, MA (US); Dennis Arthur Ausiello, Boston, MA (US); Thomas E. Devlin, Boston, MA (US)

(21) Appl. No.: 17/602,981

(22) PCT Filed: Apr. 10, 2020
(86) PCT No.: PCT/US2020/027818

§ 371 Date: Oct. 11, 2021

Related U.S. Application Data
(60) Provisional application No. 62/833,366, filed on Apr. 12, 2019.
(60) Provisional application No. 62/908,239, filed on Sep. 30, 2019.
(60) Provisional application No. 62/984,697, filed on Mar. 3, 2020.

(51) Int. Cl. A61M 21/00; B05B 17/06; B05B 12/12

(52) U.S. Cl. CPC A61M 21/00; B05B 17/0653; A61M 2205/123; A61M 2021/0016; B05B 12/12

(57) ABSTRACT

An apparatus to provide scent or other substances (e.g., active agent) includes a thin profile delivery apparatus that includes an actuator (e.g., nebulizer) and drive circuit to generate a spray, aerosol or vapor. The apparatus may have a slot to removably a media cartridge that carries the scent or other substances. The apparatus may include two contacts (e.g., buttons), opposed to one another across a dimension of the apparatus. Activation may require both engagement of both contacts, for instance concurrently, or even successive engagement of both contacts, in quick succession. Activation may further require a correct orientation of the apparatus. The apparatus may have a form factor similar to a conventional credit or debit card, and may be pliable or pliably resilient. The delivery apparatus can be used in entertainment (e.g., food or cooking programming), and in healthcare (e.g., control of metabolism).

### FIELD

This disclosure generally relates to providing or dispensing scents, therapeutic and other substances, and in particular to related delivery apparatus having a thin profile, and related systems, methods, and articles of manufacture including media cartridges.

### BACKGROUND

### Description of the Related Art

Scents may be provided a pleasant sensory experience to individuals, whether to mask offensive odor or to create a pleasing environment. Scents and other substances may additionally or alternatively be provided to achieve a therapeutic effect.

Although some systems and devices have been proposed for attempting to provide olfactory sensations to users (see, for instance, U.S. Pat. Nos. 8,050,545; 8,032,014; 6,654,664; and 6,803,987), they have proven inadequate as mobile, personal, targeted and effective delivery systems that may be used to alter behavior.

As explained herein, new approaches that effectively deliver therapeutic and other substances in order to elicit a physiological response are desirable.

### BRIEF SUMMARY

The delivery of scents and/or other substances (e.g., active substances) to the nasal epithelium, generally involves the delivery of dry or liquid formulations to the nose via a nebulizer, metered dose inhaler, or dry powder inhaler. These delivery modalities conventionally involve spraying or sniffing active substances directly into the nose via the nostrils or nasal vestibule.

Naturally, delivery of odorants (i.e., scent molecules) to the nasal epithelium occurs in two ways. The first, ortho-nasal scent delivery, occurs by sniffing odorants in the atmosphere, e.g., directly via the nostrils or nasal vestibule. The second, retro-nasal scent delivery, occurs by the natural diffusion and convection of odorants in the mouth into the nasal passages via the oropharynx. This latter delivery is referred to as retro-nasal olfaction, and is promoted by exhalation.

It has recently been found that many people who cannot perceive scent via ortho-nasal olfaction, can actually perceive scent or flavor via retro-nasal olfaction. The surprising “special capacity” of retro-nasal olfaction relates to the fact that the human oropharynx is supremely well designed to bring odorants in the mouth into the nasal passages. As a consequence, flavor perception plays a critical role in the regulation of human metabolism. Humans develop likes and cravings for certain foods as a consequence of experiencing the metabolic effects of these foods, and associating these effects with flavor images in their brains. Eventually, these images, as memories (the olfactory nerve links olfactory receptors in the nose with the seat of long-term memory, the hippocampus) drive food interests and cravings that lead to humans receiving the metabolic effects they enjoy.

Recently, human and animal studies have found that simply perceiving the scent of certain foods, like chocolate or the aroma of roasted coffee beans, can trigger metabolic effects that heretofore have been believed to occur only on the ingestion of chocolate or coffee. This surprising finding, combined with the discovery of the general efficacy of retro-nasal olfaction versus ortho-nasal olfaction, opens up a completely new opportunity for active substance (e.g., drugs, and various scent molecules that have until now principally been understood to relate to food and flavor perception) delivery to the nose.

Described herein are new apparatus, methods and articles for delivery of scents and/or other substances to the nose. The described apparatus, methods and articles can be employed for masking odors, providing a pleasing environment, and/or to achieve a therapeutic effect (e.g., up- and down-regulation of human and other animal metabolism; decongestion).

As also described herein, delivery apparatus are described which allow the portable, discrete delivery of scents and other substances, enhancing or efficiency of delivery to humans and other animals. Advantageously, the delivery apparatus is configured to be portable, allowing the user to have the benefit of delivery on demand, in a wide variety of environments. The apparatus may, for example have a form factor similar to a conventional credit or debit card, having a housing comprises of a pair of major walls or faces in opposed relation across a thickness, and spaced apart from one another by a relatively thin peripheral wall. The apparatus may, for example, be resiliently compliant, similar to the resiliency of conventional credit or debit cards. In some implementations, the apparatus may include one, two or more hinges (e.g., living hinge, butt or mortise hinge, piano hinge, flush hinge). The apparatus may easily fit in a pocket or even a conventional wallet.

The apparatus may include a nebulizer, for example a nebulizer comprising a vibration surface or element (e.g., screen, plate) and a piezo-electric element, solenoid, or an electric motor physically (e.g., mechanically, magnetically) coupled to move (e.g., oscillate, pivot, rotate) the vibration surface or element and thereby cause dispersion of the scent media in the interior of the vessel, for instance as a spray.

The apparatus optionally includes at least one outlet (e.g., port) that provides communication between at least a portion of an interior of the housing and an exterior thereof. The outlet may be positioned and oriented so that a spray or other distribution of scent media is directed outwardly of the housing to be sampled by a human end user. A protective screen may overlie the outlet, preventing ingress of fingers or debris while allow egress of scent particles.

The apparatus include one or more slots in which media cartridges holding scent media may be received or removably received at least partially into the interior of the housing. The slot may, for example, be formed in a peripheral or side wall of the housing. The slot may be dimensioned and shaped to closely receive media cartridges and position a portion of the media cartridge (e.g., scent media carrying portion) between the outlet and the vibration surface or element. In this respect, the housing may include a media cartridge receptacle area between the outlet and the vibration surface or element, sized and dimensioned to receive a portion of the media cartridge. Alternatively, the apparatus may have a permanent piece of media (e.g., scent media) located therein, the apparatus disposable after the scent media is spent.

The apparatus includes a circuit that is operable to deliver power to the piezo-electric element, solenoid, or electric motor to move (e.g., oscillate, pivot, rotate) the piezo-electric element, solenoid, or electric motor at one or more desired frequencies (e.g., relatively high frequencies). The apparatus includes a power source, which may take the form of primary or secondary chemical batteries, ultra-capacitor cells, and/or fuel cells. The apparatus may include a manual switch, a capacitive switch, and inductive switch to allow the apparatus to be turned ON and OFF via the circuit. The apparatus may include a proximity sensor (e.g., passive infrared (PIR) proximity sensor, motion sensor, accelerometer(s), facial recognition sensor (e.g., cameras and dot projector)) to allow the apparatus to be turned ON and OFF via the circuit, in response to a presence of an individual, touch, or motion).

A delivery apparatus may be summarized as including: a housing having a pair of major faces, the major faces opposed to one another across a thickness of the housing, the housing having an interior between the major faces, a media cartridge receiver sized to receive at least a portion of a media cartridge thereby, and at least one port that provides fluid communications between at least a portion of the interior of the housing and an external environment external to an exterior of the housing; a nebulizer housed in the housing, the nebulizer including a vibration member and an actuator coupled to cause the vibration member to vibrate at one or more frequencies; a pair of contacts manually engageable by a user from the exterior of the housing; a drive circuit comprising at least one switch responsive to manual engagement of the contacts, the drive circuit coupled to drive the actuator in response an occurrence of at least one condition, the at least one condition including at least a manual engagement of each of the pair of contacts at least within a defined time threshold of one another.

The pair of contacts may, for example, comprise a pair of buttons, the buttons opposed to one another across a dimension of the housing.

The pair of contacts may, for example, comprise a pair of buttons, the buttons opposed to one another across a longitudinal axis of the housing.

The pair of contacts may, for example, comprise a pair of buttons, the buttons each positioned on a respective peripheral side edge of the housing.

The pair of contacts may, for example, comprise a pair of buttons, the buttons each extending along a majority of a length of a respective peripheral side edge of the housing.

The pair of contacts may, for example, comprise a pair of peripheral side edges of the housing.

The at least one switch may, for example, comprise a pair of momentary contact switches, each of the momentary contacts switches response to manual engagement of a respective one of the contacts.

The drive circuit may, for example, comprise at least one sensor response to a force applied at the contacts.

The drive circuit may, for example, comprise at least one sensor response to contact with bodily tissue at the contacts.

The defined time threshold may require manual engagement of a second one of the contacts to be manually engaged concurrently with manual engagement of a first one of the contacts to be manually engaged.

The defined time threshold may require manual engagement of a second one of the contacts to be manually engaged simultaneously with manual engagement of a first one of the contacts to be manually engaged.

The defined time threshold may require manual engagement of a second one of the contacts to be manually engaged within less than 0.5 seconds of manual engagement of a first one of the contacts to be manually engaged.

The drive circuit may comprise at least one sensor response to an orientation of the delivery apparatus.

The delivery apparatus may comprise at least one sensor responsive to an orientation of the delivery apparatus comprises at least one of an accelerometer or a proximity sensor.

The at least one condition may include the orientation of the delivery apparatus being within a defined range of orientations at least within a threshold period of the manual engagement of each of the pair of contacts at least within the defined time threshold of one another.

The at least one condition may include at least two successive manual engagements of each of the pair of contacts at least within a defined successive engagement time threshold.

The media cartridge receiver may, for example, comprise a slot in the housing and a gasket, the gasket having an opening sized and shaped to closely receive a neck of the media cartridge.

A front one of the major faces of the housing may be planar, and a rear one of the major faces of the housing may be non-planar.

The media cartridge receiver sized to closely, removably, receive at least a portion of the media cartridge thereby.

An inner portion of a fluid path in the housing may have a hydrophilic surface or a hydrophilic coating or film. An inner portion of a fluid path in the housing may have a rough surface to increase a turbulence in a fluid flow.

A delivery system may be summarized as including: a media cartridge; and a delivery apparatus, the delivery apparatus comprising: a housing having a pair of major faces, the major faces opposed to one another across a thickness of the housing, the housing having an interior between the major faces, a media cartridge receiver sized to receive at least a portion of the media cartridge thereby, and at least one port that provides fluid communications between at least a portion of the interior of the housing and an external environment external to an exterior of the housing; a nebulizer housed in the housing, the nebulizer including a vibration member and an actuator coupled to cause the vibration member to vibrate at one or more frequencies; a pair of contacts manually engageable by a user from the exterior of the housing; a drive circuit comprising at least one switch responsive to manual engagement of the contacts, the drive circuit coupled to drive the actuator in response an occurrence of at least one condition, the at least one condition including at least a manual engagement of each of the pair of contacts at least within a defined time threshold of one another.

At least an inner portion of the media cartridge or of a fluid path in the housing may have a hydrophilic surface or a hydrophilic coating or film. An inner portion of the media cartridge or of a fluid path in the housing may have a rough surface to increase a turbulence in a fluid flow. The media cartridge may contain a surfactant added to the media carried in the media cartridge.

A method of operation in a delivery apparatus is described, the delivery apparatus including: a housing having a pair of major faces, the major faces opposed to one another across a thickness of the housing, a nebulizer, a pair of contacts manually engageable by a user from the exterior of the housing, a drive circuit coupled to drive the nebulizer in response an occurrence of at least one condition. The method may be summarized as including: detecting a manual engagement of a first one of the pair of contacts; detecting a manual engagement of a second one of the pair of contacts; determining if the manual engagement of the second one of the pair of contacts occurred within a defined time threshold of the manual engagement of the second one of the pair of contacts; and operating the nebulizer based, at least in part, on the determination of whether the manual engagement of the second one of the pair of contacts occurred within a defined time threshold of the manual engagement of the second one of the pair of contacts.

The pair of contacts comprise a pair of buttons, the buttons opposed to one another across a longitudinal axis of the housing, the buttons each positioned on a respective peripheral side edge of the housing, and detecting a manual engagement of a first one of the pair of contacts may include detecting a depression of a first one of the pair of buttons, and detecting a manual engagement of a second one of the pair of contacts includes detecting a depression of a second one of the pair of buttons.

The pair of contacts may comprise a pair of peripheral side edges of the housing, and detecting a manual engagement of a first one of the pair of contacts may include detecting a force applied to a first one of the side edges, and detecting a manual engagement of a second one of the pair of contacts includes detecting a force applied to a second one of the side edges.

The pair of contacts may comprise a pair of peripheral side edges of the housing, and detecting a manual engagement of a first one of the pair of contacts may include detecting a depression of a first one of the side edges, and detecting a manual engagement of a second one of the pair of contacts includes detecting a depression of a second one of the side edges.

The delivery apparatus may include a pair of momentary contact switches, and detecting a manual engagement of a first one of the pair of contacts may include detecting an activation of a first one of the pair of momentary contact switches, and detecting a manual engagement of a second one of the pair of contacts includes detecting an activation of a second one of the pair of momentary contact switches.

Determining if the manual engagement of the second one of the pair of contacts occurred within a defined time threshold of the manual engagement of the second one of the pair of contacts may include determining whether the manual engagement of a second one of the contacts occurs concurrently with manual engagement of a first one of the contacts.

Determining if the manual engagement of the second one of the pair of contacts occurred within a defined time threshold of the manual engagement of the second one of the pair of contacts may include determining whether the manual engagement of a second one of the contacts occurs simultaneously with manual engagement of a first one of the contacts.

Determining if the manual engagement of the second one of the pair of contacts occurred within a defined time threshold of the manual engagement of the second one of the pair of contacts may include determining whether the manual engagement of a second one of the contacts occurs within less than 0.5 seconds of manual engagement of a first one of the contacts to be manually engaged.

Determining if at least two successive manual engagements of each of the pair of contacts has occurred at least within a defined successive engagement time threshold, and wherein operating the nebulizer is further based, at least in part, on the determination of whether at least two successive manual engagements of each of the pair of contacts has occurred at least within the defined successive engagement time threshold.

The drive circuit may include at least one sensor response to an orientation of the delivery apparatus. The method may further comprise: determining whether an orientation of the delivery apparatus is within a defined range of orientations at least within a threshold period of the manual engagement of each of the pair of contacts.

Operating the nebulizer based, at least in part, on the determination of whether the manual engagement of the second one of the pair of contacts occurred within a defined time threshold of the manual engagement of the second one of the pair of contacts may include operating the nebulizer in response to both: i) the manual engagement of the second one of the pair of contacts occurred within a defined time threshold of the manual engagement of the second one of the pair of contacts, and ii) orientation of the delivery apparatus is within a defined range of orientations at least within a threshold period of the manual engagement of each of the pair of contacts.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is an front, top, left side isometric view of a delivery apparatus that is operable to deliver the scent or other substance, according to one illustrated implementation.

FIG. 2 is an exploded view a delivery apparatus and removable media cartridge that is operable to nebulize and deliver scent or other substance, according to one illustrated implementation.

FIG. 3 is a schematic diagram of a piezo electric actuator and circuit coupled and operable to drive the piezo-electric actuator to oscillate at a defined frequency or frequencies to produce an aerosol or nebulized cloud, according to one illustrated implementation.

FIG. 4 is a front plan view of a delivery apparatus that is operable to nebulize and deliver a scent or other substance, along with a replaceable media cartridge that is carries scent media or other media to be delivered, according to another illustrated implementation.

FIG. 5 is a right side elevational view of the delivery apparatus and replaceable media cartridge of FIG. 4.

FIG. 6 is a rear plan view of the delivery apparatus and replaceable media cartridge of FIG. 4.

FIG. 7 is a top edge elevational plan view of the delivery apparatus of FIG. 4.

FIG. 8 is a bottom edge elevational plan view of the delivery apparatus of FIG. 4.

FIG. 9 is a cross-sectional view of the delivery apparatus and replaceable media cartridge of FIG. 4 taken along section line A-A.

FIG. 10 is a cross-sectional view of the delivery apparatus and replaceable media cartridge of FIG. 4 taken along section line B-B.

FIG. 11 is a cross-sectional view of the delivery apparatus and replaceable media cartridge of FIG. 4 taken along section line C-C.

FIG. 12 is an exploded view the delivery apparatus and replaceable media cartridge of FIG. 4.

FIG. 13 is a front, isometric view of the delivery apparatus of FIG. 4 and a collapsible scent concentrator, according to one illustrated implementation, in an expanded configuration.

FIG. 14 is a front, isometric view of the collapsible scent concentrator of FIG. 13 in a collapsed configuration.

FIG. 15 is a front, isometric view of the delivery apparatus of FIG. 4 and a collapsible scent concentrator in an expanded configuration.

FIG. 16 is a front, isometric view of the delivery apparatus and collapsible scent concentrator of FIG. 15 in a collapsed configuration.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with microcontrollers, piezo-electric devices, air moves, power supplies such as DC/DC converters, wireless radios (i.e., transmitters, receivers or transceivers), computing systems including client and server computing systems, and networks (e.g., cellular, packet switched), as well as other communications channels, have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Reference herein to two elements “facing” or “facing toward” each other indicates that a straight line can be drawn from one of the elements to the other of the elements without contacting an intervening solid structure. Reference herein to two elements being “directly coupled” indicates that the two elements physically touch with no intervening structure between.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The headings and Abstract of the Disclosure provided herein are for convenience only and do not interpret the scope or meaning of the embodiments.

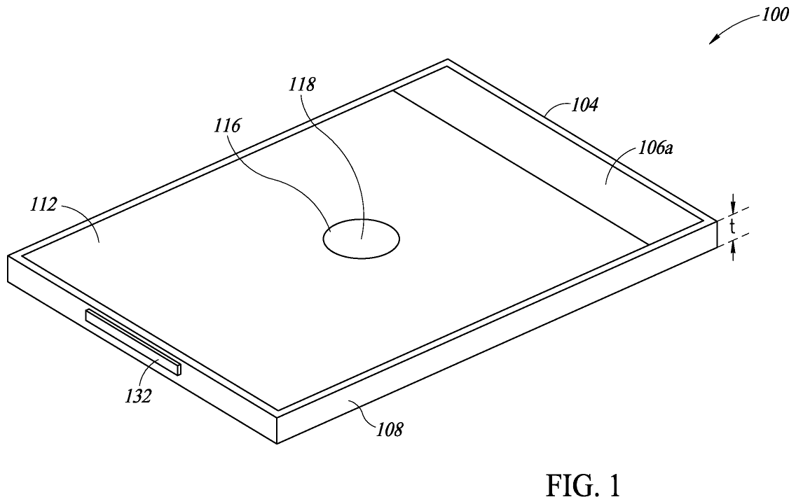

FIG. 1 shows a delivery apparatus 100 that is operable to deliver the scent or other substance, according to one illustrated implementation.

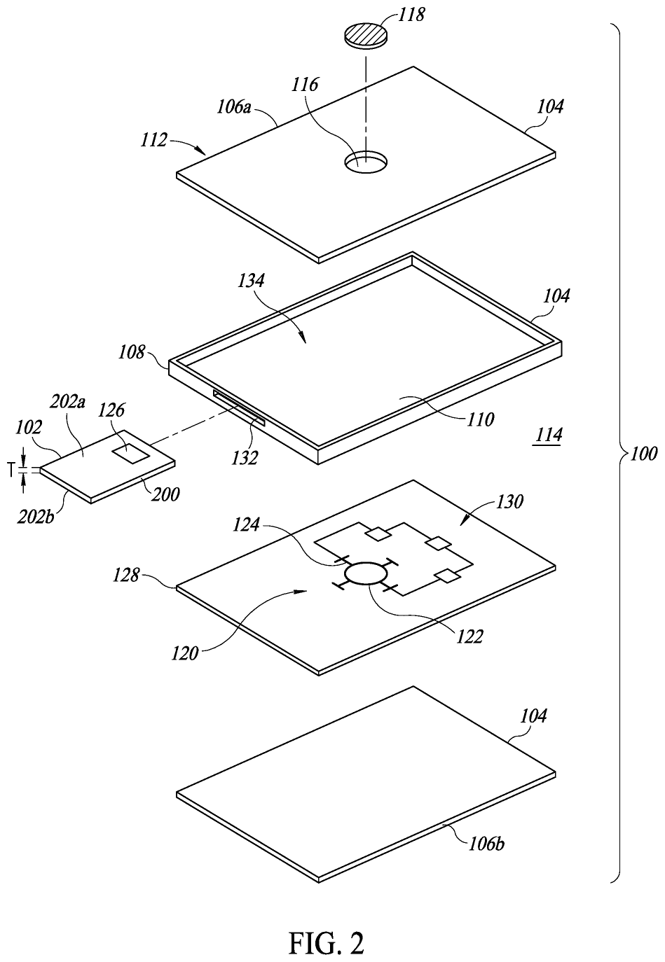

FIG. 2 show the delivery apparatus 100 in exploded form, along with a removable media cartridge 102 that is operable to deliver the scent or other substances, according to one illustrated implementation.

With reference to FIGS. 1 and 2, the delivery apparatus 100 includes a housing 104, the housing having a pair of major walls or faces 106a, 106b in opposed relation to one another across a thickness t, and spaced apart from one another by a relatively thin peripheral wall 108. The major walls or faces 106a, 106b and peripheral wall 108 define an interior 110 (FIG. 2) of the housing 104, and separate the interior 110 of the housing 104 from the exterior 112 of the housing and external environment 114. The housing 104 may include two inseparable parts, for example heat welded, RF welded or adhesively fixed together. Such may provide a water resistant or water-tight housing 104. Alternatively, the housing 104 may include two separable parts, with a seam therebetween, for example to allow the power source to be replaced. The parts may be include threads or other coupling structures (not shown) allowing the parts to be screwed apart and screwed together. One or more gaskets (not shown) can provide hermetical sealing of the interior 110 of the housing 104 from an exterior 112 thereof.

The delivery apparatus 100 optionally includes at least one outlet 116 (e.g., port) that provides communication between at least a portion of the interior 110 of the housing 104 and the exterior 112 thereof. The outlet 116 forms part of the fluidly communicative path 108 for the fluid or vapor or aerosol to be transferred from the interior 110 to the external environment 114. The outlet 106 may be positioned and oriented so that a spray or other distribution of scent media is directed outwardly of the housing 104 to be sampled by a human end user. A protective screen 118 may overlie the outlet 116, having a mesh size that prevents ingress of fingers or debris while allowing egress of scent particles or other substances in a spray, vapor or aerosol form.

The delivery apparatus 100 may include a nebulizer 120. The nebulizer 120 may include comprising a vibration surface or element 122 (e.g., mesh, plate) and an actuator 124 (e.g., a piezo-electric element, solenoid, or an electric motor) physically (e.g., mechanically, magnetically) coupled to move (e.g., oscillate, pivot, rotate) the vibration surface or element 122 and thereby cause dispersion of the scent media in the interior 110 of the housing 104, for instance as a spray, vapor or aerosol.

The actuator 124 may be used to turn media into the spray, vapor or aerosol that is transmitted from the interior 110 of the housing 104 to the external environment 114. In some implementations, for example, the actuator 124 is a piezo-electric element or other transducer that is physically coupled to oscillate a metal mesh 122 to generate a mist. The piezo-electric element or other transducer may oscillate at a frequency of about 175 kHz±5 kHz that is sufficient to atomize media held within the housing 104, for instance held on the media cartridge 102. The frequency of oscillation of such a transducer may be increased or decreased depending upon the properties of the media or other materials. In such an implementation, that transducer may form an annular ring with a metal-mesh included within a center portion of the transducer. As such, the media may be transported to the metal mesh, via, for example, capillary action, where it is atomized into the vapor or aerosol as a result of the oscillation of the transducer. The vibration surface or element 122 of the nebulizer 122 may be located towards a bottom face 106b of the housing 102 where the port 116 is positioned in the top face 106b of the housing 102. A media 126 bearing portion of the media cartridge 102 may be interposed between the vibration surface or element 122 and the port 116 when the media cartridge 102 is correctly positioned in the housing 104, as discussed below.

The delivery apparatus 100 may include a circuit board 128 (FIG. 2). The circuit board 128 may carry a circuit 130 that is operable to deliver power to the piezo-electric element, solenoid, or electric motor to move (e.g., oscillate, pivot, rotate) the piezo-electric element, solenoid, or electric motor at one or more desired frequencies (e.g., relatively high frequencies). In some implementations, the circuit board 128 may also carry the piezo-electric element, solenoid, or electric motor. Alternatively, the circuit 128 and/or the piezo-electric element, solenoid, or electric motor may be mounted or attached to one or both of the major walls or faces 106a, 106b of the housing 104.

The delivery apparatus 100 includes one or more slots 132 in which media cartridges 102 holding media 126 (e.g., scent media, for instance in liquid form) may be received or removably received at least partially into the interior 110 of the housing 104. The slot 132 may, for example, be formed in a peripheral or side wall 108 of the housing 104. The slot 132 may be dimensioned and shaped to closely receive media cartridges 102 and position a portion of the media cartridge 102 (e.g., scent media 126 carrying portion) between the outlet 116 and the vibration surface or element 122. In this respect, the housing 104 may include a media cartridge receptacle area 134 (FIG. 2) between the outlet 116 and the vibration surface or element 122, sized and dimensioned to receive a portion of the media cartridge 102. Alternatively, the delivery apparatus 100 may have a permanent piece of scent media located therein, the delivery apparatus 100 disposable after the scent media is spent.

The delivery apparatus 100 may, for example, be resiliently compliant, similar to the resiliency of conventional credit or debit cards. For example, the delivery apparatus 100 may be formed of a plastic similar to that used in conventional credit or debit cards (e.g., polyvinyl chloride). The delivery apparatus 100 may have a thickness equal to 1 to 5 times the thickness of conventional credit or debit cards (e.g., 1 to 5 times 0.03 inches, 0.76 millimeters or 30 mil). The circuit board 128 may be a flexible printed circuit board (PCB). The housing 104 and/or circuit board 128 may have one or more flexures formed therein, to facilitate flexibility without plastic failure. Additionally or alternatively, the delivery apparatus 100 may have a rubber or silicone sleeve, for example over-molded on all or a portion of the housing 104.

In some implementations, the delivery apparatus 100 may include one, two or more hinges (e.g., living hinge, butt or mortise hinge, piano hinge, flush hinge). Thus, the housing 104 may comprise two or more portions, each hinged to pivot with respect to another portion. Advantageously, the delivery apparatus 100 is configured to be portable, allowing the user to have the benefit of delivery on demand, in a wide variety of environments. The delivery apparatus 100 may, for example have a form factor similar to a conventional credit or debit card. The apparatus may easily fit in a pocket or even a conventional wallet.

The media cartridge 102 may include a substrate 200 having a pair of major faces 202a, 202b (collectively 202), the major faces 202 opposed across a thickness T of the media cartridge 102 from one another. The substrate 200 of the media cartridge 102 carries at least one piece of consumable media 126. The consumable media 126 may be in a form that releases at least one of scent molecules, active substances, or other substances as a spray, a vapor and/or an aerosol when subjected to oscillation at or above at least one defined frequency. The substrate 200 may be sized and shaped to be closely, removably received at least partially in a cartridge receiver slot 132 of the delivery apparatus 100. The scent media 126 is in the form of a liquid, a powder, a gel, a suspension, or a volatile organic. The media cartridge 102 may formed of any of a large variety of materials that are impervious to the particular media to be carried. Suitable materials may, for example include various polymers, plastics, and in particular acrylics. The media cartridge 102 may advantageously be transparent or at least translucent, which allows a level of media within the cartridge to be visually assessed.

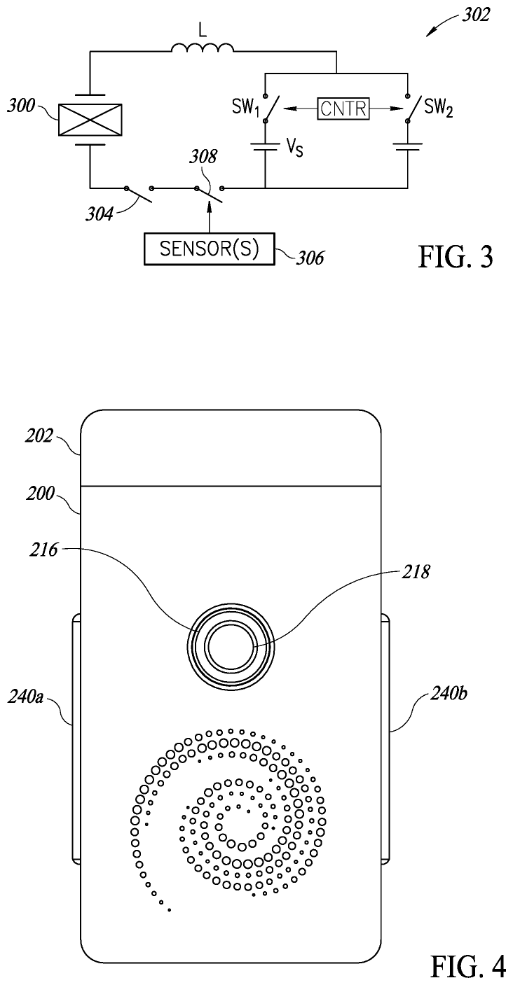

FIG. 3 shows a piezo-electric element 300 and circuit 302 coupled and operable to drive the piezo-electric actuator 300 of the delivery apparatus 100 to oscillate at a defined frequency or frequencies to produce an aerosol or nebulized cloud, according to one illustrated implementation.

As noted, the delivery apparatus 100 includes a circuit 302 that is operable to deliver power to the piezo-electric element 300 at one or more desired frequencies (e.g., relatively high frequencies). The circuit 302 includes one or more power source Vs, which may take the form of primary or secondary chemical batteries, ultra-capacitor cells, and/or fuel cells. The delivery apparatus 100 may, for example, include a set of electrical contacts (not shown) accessible from the exterior 112 of the housing 104 or inductive charger element (not shown) to allow recharging of the power source Vs.

The circuit 302 may include a pair of switches SW₁, SW₂ coupled between voltage source(s) Vs and the piezo-electric element 300. The circuit 302 includes a switch controller 304 that, when activated, controls the switches SW₁, SW₂. For example, the switch controller 304 can apply voltage signals to the gates of a pair of transistors. The switch controller 304 may, for example, alternate between opening and closing a first switch SW₁ and concurrently alternate between closing an opening the second switch SW₂, in opposition fashion to generate an alternating voltage at the piezo-electric element 300. The switch controller 304 may take the form of an integrated circuit. The circuit may include an inductor L. While one example alternating or resonant circuit is illustrated, the delivery apparatus 100 may employ any of a large variety of alternating or resonant circuits to drive the piezo-electric element 300 or other actuator in oscillation. Thus, the claims should not be limited to a particular drive circuit unless the specific elements of the drive circuit are positively recited in the claims.

For example, the delivery apparatus 100 may optionally include a control system that may include a microcontroller. A suitable microcontroller may take the form of an 8-bit microcontroller with in-system programmable flash memory, such as the microcontroller commercially available from Atmel Corporation under designation ATMEGA48/88/168-AU. The microcontroller executes a program stored in its memory, and sends signals to control the various other components, such as, for example, the vibration surface or element 122 of nebulizer 120 Control signals may, for instance be pulse width modulated (PWM) control signals. Otherwise, control signals may take on any of a large variety of forms. For instance, the microcontroller may simply complete a circuit that powers the nebulizer 120.

The delivery apparatus 100 may optionally include a visual indicator to indicate when the delivery apparatus 100 is operating or turned ON. The visual indicator may, for example take the form of a single light emitting diode (LED), although in various implementations the visual indicator may take any of a large variety of forms. The LED may be capable of emitting one, two or more distinct colors. The visual indicator may also indicate other information or conditions, for instance the visual indicator 162 may flash in response to an occurrence of an error condition. A pattern of flashes (e.g., number of sequential flashes, color of flashes, number and color of sequential flashes) may be used to indicate which of a number of possible error conditions has occurred.

The circuit 302 may include a manually operated switch 304 which powers the delivery apparatus 100 ON and OFF. The manually operated switch 304 can take a variety of forms, for example a contact switch, toggle switch, momentary contact switch, rocker switch, capacitive switch, or inductive switch. The circuit 302 may additionally or alternatively include one or more sensors 306 coupled to one or more switches 308 which cause the delivery apparatus 100 to operate (e.g., dispense spray, vapor, aerosol). The sensor(s) 306 may take a variety of forms, for example, a proximity sensor (e.g., passive infrared (PIR) proximity sensor), motion sensor, one-, two- or three-axis accelerometer(s), facial recognition sensor (e.g., cameras and dot projector), Reed sensor, that may cause the nebulizer to produce spray, vapor or aerosol in response to a presence of an individual, touch of the delivery apparatus 100, motion (e.g., upward movement, downward movement) of the delivery apparatus 100, orientation (e.g., tilting, upright) of the delivery apparatus 100.

Activation may be synchronized with the delivery of audio, video, or audiovisual media. For example, a smartphone or digital assistance (e.g., Amazon Alexa®, Google Home®, Apple HomePod®) can cause activation of flavorful droplets inside a vessel that a consumer can experience in coordination with the delivery or experience of other digital media, e.g., music, film, video games, virtual reality (VR), augmented reality (AR), etc.

The scent media 126 may take any of a variety of forms, for instance including but limited to fluids, powders, gels, suspensions, wax, oils, essential oils, and/or volatile compounds. In some implementations, scent or other substances or compositions are advantageously formulated as or in readily-soluble water droplets. The readily-soluble water droplets have a median size range of approximately 2 microns to approximately 50, 20, or 10 microns. The substances can be dissolved, if water soluble, directly in the water droplets. The substances (e.g., active substances) can, for example if not water soluble, be encapsulated inside, or otherwise formulated as, micelles, micro-emulsions, emulsions, liposomes, nanoparticles or other kinds of colloids. These colloids do not have a size larger than 500 nm, and optimally 200 nm or smaller. The small size of these colloids permits the nebulization of the droplets without destroying the colloids or otherwise impeding (e.g., clogging) the nebulizer (e.g., ultrasound transducer).

The composition, apparatus, methods and articles described herein have various useful benefits. The delivery of active substances or compositions (e.g., odorants or flavorful molecules of some kind, or other more traditional therapeutics) that up- and down-regulate metabolism, can be achieved with less than 1 gram of ingested active substance or composition. That is, the approaches described herein can deliver active substances to olfactory and/or taste receptors, producing physiological benefit (e.g., up-regulating and, or down-regulating human metabolism), while delivering almost no active substance to the gastro-intestinal (GI) tract. Second, for the purposes of delivery solely to the nose, as in the delivery of decongestants for the common cold or other respiratory dysfunction, the approaches described herein can produce greater physiological effect per nominal dose delivered to the nose than any other approach known by applicants, as in a spray or respiration from the environment into the nose.

In some implementations, the media cartridge 102 may be used to hold and contain one or more media 126 (e.g., scent particles or molecules, other substances, active substances) as a fluid or other material (e.g., powder, gel, colloidal suspension) that carries the scent particles or scent molecules, or other substances. In some implementations, for example, the media cartridge 102 may be sized and dimensioned to hold up to 100 mL of the fluid 110. In some implementations, the media cartridge 102 may be sized and dimensioned to hold a maximum amount of the fluid 110 that is less than 100 mL (e.g., 5 mL, 10 mL, 20 mL, 40 mL, or 50 mL). The scent media 126 may, for example, be any liquid or other material that is, or that carries, the scent particles or molecules or other substances (e.g., active substance(s)) that are released when the fluid transitions to a vapor or aerosol and is released into the exterior or external environment.

The vapor or aerosol may advantageously comprise readily-soluble water droplets have a median size range of approximately 2 microns to approximately 50, 20, or 10 microns. The vapor or aerosol may result in a physiological response from some users when those users encounter the scent or other substance(s) transported by the vapor or aerosol.

FIGS. 4, 5, 6, 7, 8, 9, 10, 11, and 12 show a delivery apparatus 200 that is operable to deliver media (e.g., scent or other substances) in the form of an aerosol, vapor, or cloud, according to one illustrated implementation. FIGS. 4, 5, 6, 9, 10, 11, and 12 also show a replaceable media cartridge 202 that is selectively dockable to the delivery apparatus 200 to provide media thereto, for example in a liquid form, for dispensation from the delivery apparatus 200 as an aerosol, vapor, or cloud.

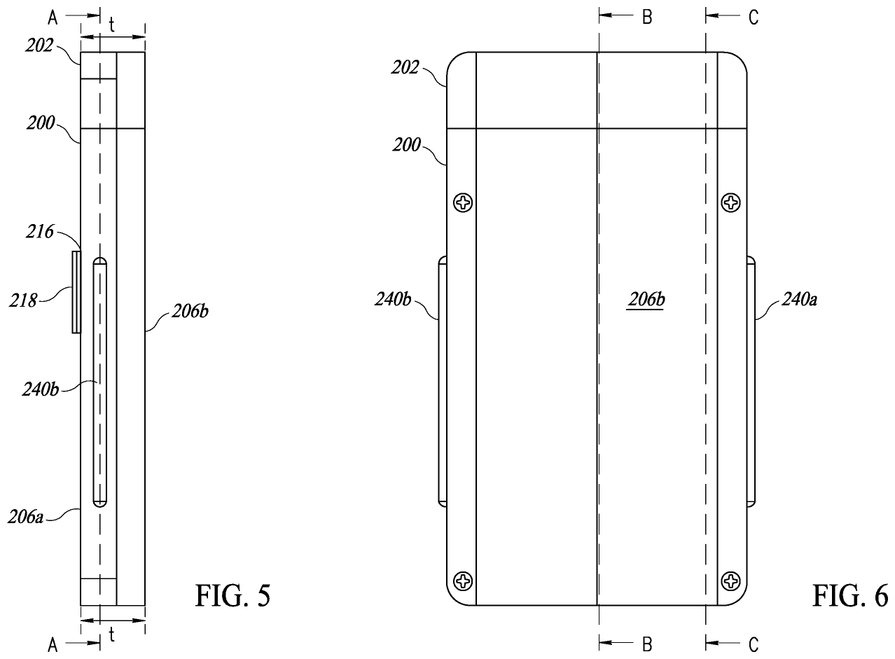

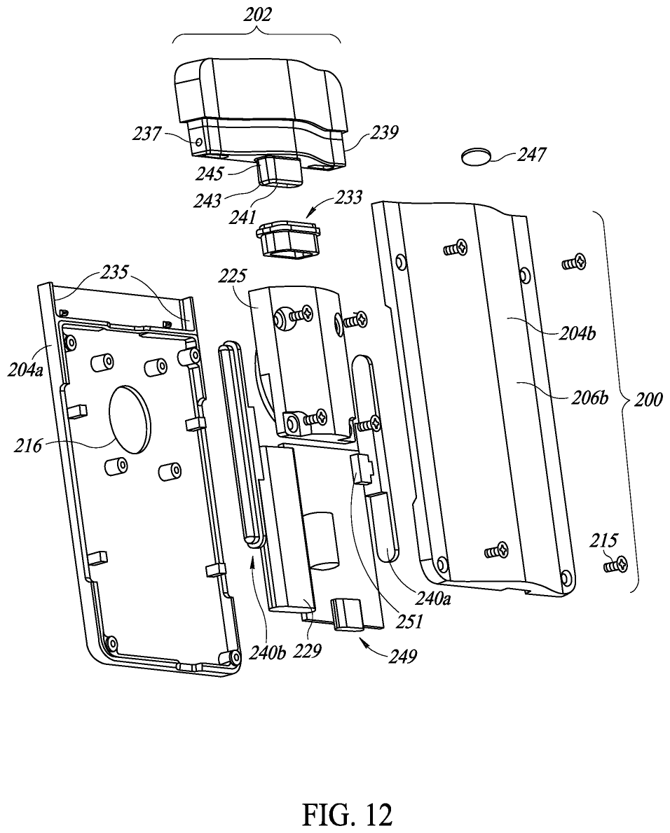

The delivery apparatus 200 may include a housing 204, for example comprised from two pieces 204a, 204b. The two pieces 204a, 204b provide a pair of major walls or faces 206a, 206b in opposed relation to one another across a thickness t (FIGS. 5 and 11), and spaced apart from one another by a relatively thin peripheral wall 208. A front one of the major walls or faces 206a may, for instance, be planar or flat. A rear one of the major walls or faces 206b may, for instance, be nonplanar or not flat, having for instance a convex shape (e.g., arcuate shape in profile) or a peaked shape (e.g., A shape in profile), with an outwardly extending center portion, to better be accommodated by a human hand. The major walls or faces 206a, 206b and peripheral wall 208 define an interior 210 (FIG. 9) of the housing 204, and separate the interior 210 of the housing 204 from the exterior 212 (FIG. 9) of the housing and external environment. The pieces 204a, 204b may be fastened together via fasteners 215 (only one called out, FIG. 12, e.g., screws, bolts and nuts, rivets), via detents (not shown), a weld (e.g., heat weld, RF weld, ultrasonic weld), and/or via adhesive (not shown). One or more gaskets can provide hermetical sealing of the interior 210 of the housing 204 from an exterior 212 thereof.

The delivery apparatus 200 includes at least one outlet port 216 (e.g., aperture) that provides fluid communication between at least a portion of the interior 210 of the housing 204 and the exterior 212 thereof. The outlet port 216 forms part of a fluidly communicative path for the aerosol, vapor or cloud to be transferred from the interior 210 to the external environment 214. The outlet port 216 may be positioned and oriented so that a spray or other distribution of media is directed outwardly of the housing 204 to be sampled by a human end user. A protective screen 218 may overlie the outlet port 216, having a mesh size that prevents ingress of fingers or debris while allowing egress of media (e.g., scent particles or other substances) in an aerosol, vapor or cloud form.

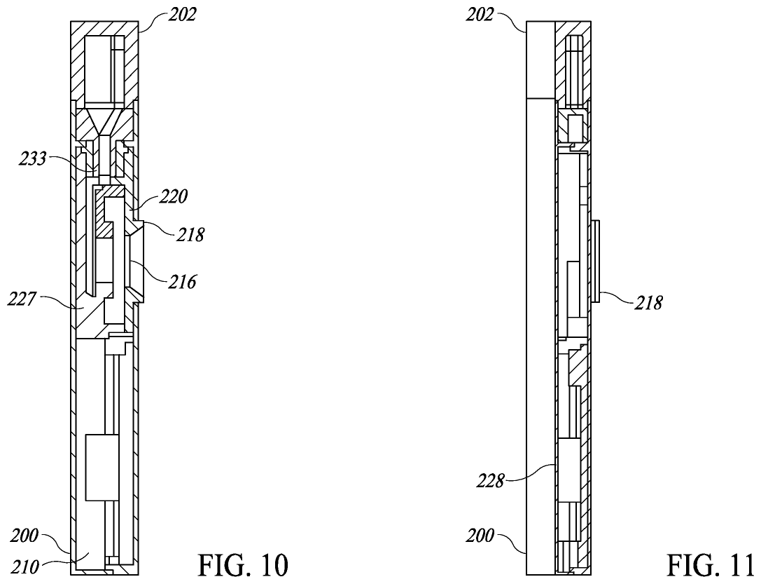

The delivery apparatus 200 may include a nebulizer 220 (FIGS. 9, 10). The nebulizer 220 may include comprising a vibration surface or element 222 (e.g., mesh, plate) (FIG. 9) and an actuator (e.g., a piezo-electric element or crystal, solenoid, or an electric motor) physically (e.g., mechanically, magnetically) coupled to move (e.g., oscillate, pivot, rotate) the vibration surface or element 222 and thereby cause dispersion of the media from the interior 210 of the housing 204 outwardly, for instance as an aerosol, vapor, cloud, or spray.

The actuator may be used to turn media into an aerosol, vapor, cloud, or spray that is transmitted from the interior 210 of the housing 204 to the external environment 214. In some implementations, for example, the actuator is a piezo-electric element or other transducer that is physically or magnetically coupled to oscillate a metal mesh 222 to generate a mist. The piezo-electric element or other transducer may oscillate at a frequency of about 175 kHz±5 kHz that is sufficient to atomize a media in the form (e.g., liquid) held within a generation chamber 225 (FIG. 12) located in the interior 210 of the housing 204, for instance a liquid delivered from the media cartridge 202. The generation chamber 225 may be delimited from the remainder of the interior 210 of the housing 204 by one or more chamber walls, and provides a space, at least in front of the nebulizer 220, in which an aerosol, vapor, or cloud can be generated or formed. The generation chamber 225 may for example be formed by a portion of one of the major walls or faces 206a and a chamber piece 227 that is positioned in the interior of the housing 204, and secured thereto, for example via fasteners (illustrated in FIG. 12, but not called out). The chamber piece 227 may be an integral part of the nebulizer 220 or provided separately from the nebulizer 220. The frequency of oscillation of such a transducer may be increased or decreased depending up on the properties of the media or other materials. In such an implementation, that transducer may form an annular ring with a metal-mesh included within a center portion of the transducer. As such, the media may be transported to the metal mesh, via, for example, capillary action, where it is atomized into the aerosol, vapor or cloud as a result of the oscillation of the transducer. The vibration surface or element 222 of the nebulizer 222 may be located towards a bottom face 206b of the housing 202 where the port 216 is positioned in the top face 206b of the housing 202.

The delivery apparatus 200 may include a circuit board 228 (FIGS. 9 and 12), battery 229 (FIG. 12), and charging port 249 (FIG. 12). The circuit board 228 may carry a circuit that is operable to deliver power to the piezo-electric element or crystal, solenoid, or electric motor to move (e.g., oscillate, pivot, rotate) the piezo-electric element or crystal, solenoid, or electric motor at one or more desired frequencies (e.g., relatively high frequencies). In some implementations, the circuit board 228 may also carry the piezo-electric element or crystal, solenoid, or electric motor. Alternatively, the circuit and/or the piezo-electric element or crystal, solenoid, or electric motor may be mounted or attached to one or both of the major walls or faces 206a, 206b of the housing 204.

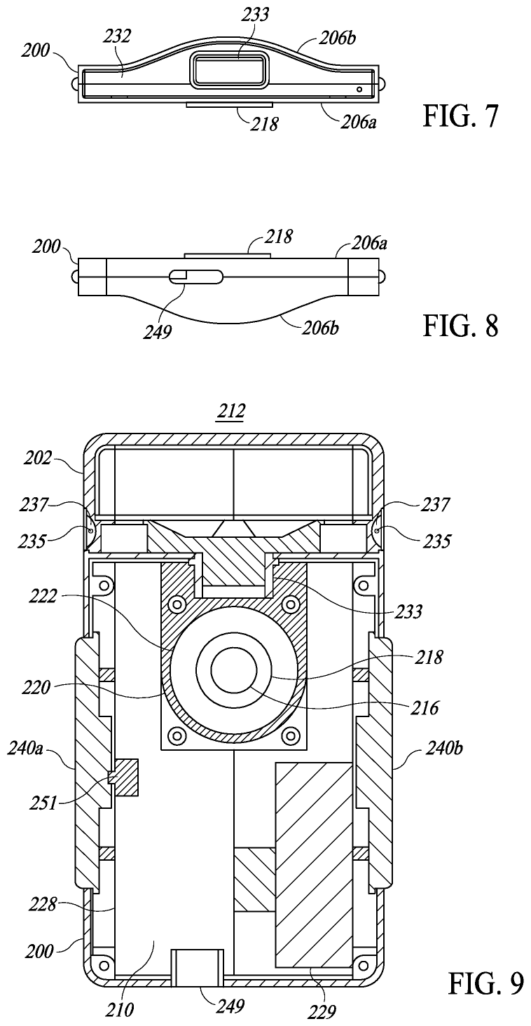

The delivery apparatus 100 may include a slot 232 (FIG. 7) in which all or a portion a media cartridge 202 holding media (e.g., scent media, for instance in liquid form) may be received or removably received providing a path for the media at least partially into the interior 210 of the housing 204, for instance into the generation chamber 225. The slot 232 may, for example, be formed in a peripheral or side wall 208 of the housing 204, for example at a top end wall of the housing as best illustrated in FIGS. 7 and 12. The delivery apparatus 100 preferably includes a gasket 233 (FIG. 12), which may for example line the slot 232, a portion thereof, or as illustrated be located at a bottom of the slot 232. The gasket 233 may optionally extend longitudinally inward and/or outward (i.e., downward or upward in view of FIGS. 4, 5, 6, 9-12) beyond the slot 232. Thus, the housing 204 may include a media cartridge receptacle area, sized and dimensioned to receive all or a portion of the media cartridge 202. The gasket 233 may be formed of a resilient, elastic material, for example silicone, to provide a hermetical seal between a portion (e.g., nozzle, neck, or outlet port) of the media cartridge 202 and a fluid path in the housing 204. The slot 232 and/or gasket 233 may be dimensioned and shaped to closely receive all or respective portions of media cartridges 202, for example providing a friction fit therebetween. Additionally, or alternatively, the housing 204, slot 232 and/or gasket 233 may include one or more retainment or latch features or structures 235 (FIGS. 9, 12) (e.g., resilient detents, latches, resilient members, prongs, keys, recesses) positioned and oriented to engage complementary retainment or latch features or structures 237 (FIGS. 9, 12) (e.g., resilient detents, latches, resilient members, prongs, keys, recesses) of the media cartridge 202.

The media cartridge 202 may comprise a media cartridge housing 200 having a pair of major faces 202a, 202b (collectively 202), the major faces 202 opposed across a thickness T of the media cartridge 202 from one another to form a reservoir therebetween. The media cartridge 202 may include an engagement portion 239 (FIG. 12), the engagement portion 239 having a shape and dimensions that are complementary to a shape and dimensions of the slot 232 of the delivery apparatus 100. The engagement portion 239 may have a perimeter that is smaller in overall dimension than an inner perimeter of the slot 232 to, for example, be closely received therein, for instance in an interference fit. The media cartridge 102 may include an outlet 241, for example located at an end 243 of a neck 245. The outlet 241 may, for example, take the form of a nozzle, or other style of outlet. The neck 245 may have an outer shape and dimensions that are complementary to an inner shape and dimensions of the gasket 233 to, for example be closely received thereby.

The media cartridge 202 may include a seal 247 (FIG. 12) that seals the outlet port at least prior to use. The seal 247 may take a variety of forms. For example, the seal 247 may comprise a frangible membrane (e.g., foil seal, plastic seal, metalized plastic seal), which can be torn, ripped, and/or punctured in preparation for use. In some implementations employing a frangible membrane, the delivery apparatus 100 may include a structure (e.g., pin, prong, blade) that is positioned to tear, rip, and/or puncture the frangible membrane as the media cartridge 202 is inserted into the slot 232 and/or gasket 233 of the delivery apparatus 100. Also for example, the seal 247 may comprise a membrane, that may or may not generally be frangible, but which is attached to the media cartridge housing via an adhesive, and which can be peeled off or peeled away in preparation for use. In some implementations, the adhesive may be reusable (e.g., pressure-sensitive adhesive), and hence the membrane may be reused to reseal the media cartridge, for instance for disposal or reuse. Membrane based approaches may advantageously not only seal media in the media cartridge, but may also provide visual and/or tactile evidence of tampering, thus constituting a tamper-evident seal. In some implementations, the destruction of or damage to the membrane may itself indicate tampering, while in other implementations the membrane may include a specific tamper-evident feature (e.g., layer of ink or colorant) that releases an ink or other colorant when torn, ripped, punctured, or removed, as visual evidence that the seal 247 has been tampered with. Also for example, the seal 247 may comprise a self-sealing seal, for instance a pair of opposed resilient members or lips, with a slit that extends between the lips, or a check valve. The opposed resilient members or lips may, for example, take the form of a silicone seal or rubber seal. In some implementations employing a self-sealing seal, the delivery apparatus 100 may include a structure (e.g., tongue, conduit, tube, straw, nozzle) that is positioned to separate the lips, for instance via insertion between the lips or applying inwardly opposed forces at points at which the lips come together, pinching the lips open, as the media cartridge 202 is inserted into the slot 232 and/or gasket 233 of the delivery apparatus 100. A self-sealing seal advantageously allows a media cartridge 202 to be removed even when the media itself has not been completely consumed, will avoiding spillage. Such can protect objects from the unspent media, and/or allow reuse of the media cartridge 202. In some implementations, a membrane seal may be employed as a tamper-evident seal, along with a self-sealing seal located under the membrane seal. Thus, a user can first inspect the membrane seal to ensure the contents have not be tampered with, remove the membrane seal, then place the media cartridge in the slot of delivery apparatus, where the self-sealing seal is opened.

As previously noted, the media cartridge 202 may include one or more retainment or latch features or structures (e.g., resilient detents, latches, resilient members, prongs, keys) 237 positioned and oriented to engage complementary retainment or latch features or structures 235 of the slot 232 and/or gasket 233.

The media cartridge 102 carries, holds or retains at least one piece of consumable media. The consumable media may be in a form that releases at least one of scent molecules, active substances, or other substances as a spray, a vapor and/or an aerosol when subjected to oscillation at or above at least one defined frequency. The media is preferably in the form of a liquid, a suspension, or a volatile organic, but can in some implementations take the form of a gel or a powder.

Portions (e.g., neck 245, outlet 241) of the media cartridge 202 and/or of the fluid path in the housing 204 may have features and/or structural characteristics to facilitate or urge media to travel from the media cartridge 202 to the nebulizer 220 (e.g., vibration surface or element 222), for instance via capillary action. For example, at least the inner portions of the various structures (e.g., media cartridge 202 and/or of the fluid path in the housing 204) may have a hydrophilic surface and/or be coated with a hydrophilic coating or film. Additionally or alternatively, at least the inner portions of the various structures (e.g., media cartridge 202 and/or of the fluid path in the housing 204) may a rough surface, for instance a dimpled surface to increase turbulence in the fluid flow. These structures may be particularly advantageous in a neck 243 or outlet 241 portion of the media cartridge 202. Additionally or alternatively, a surfactant may be added to the media carried in the media cartridge 202.

The delivery apparatus 100 may include one or more user-manipulable buttons 240a, 240b (two shown), which are accessible from the exterior of the housing 204 and are manually manipulable by a user to at least partially control operation of the delivery apparatus 100. For example, the delivery apparatus 100 may include a pair of user-manipulable buttons 240a, 240b, manipulation (e.g., depression) of which cause a change of state of one or more switches 251 (only one visible in FIGS. 9 and 12), which may start or be a prerequisite to starting the nebulizer dispensing media, for example as explained below. The delivery apparatus 100 may include one or more switches 251 associated with respective ones of user-manipulable buttons 240a, 240b. For example, the delivery apparatus 100 may advantageously include a pair of momentary contact switches 251, each of the momentary contact switches 251 positioned to be physically engaged, and hence operated by, a respective one of the user-manipulable buttons 240a, 240b, although other types of switches may be employed.

The pair of user-manipulable buttons 240a, 240b may be located opposed to one another across longitudinal axis of the housing 204, for example extending along opposed (e.g., left and right) peripheral or side wall 208. The dimensions of the delivery apparatus 100 may allow the delivery apparatus 100 to be held in a single hand of a user, who can simultaneously manipulate (e.g., depression) the pair of user-manipulable buttons 240a, 240b by, for example drawing fingers and thumb together while closing the hand. The user-manipulable buttons 240a, 240b may be biased outwardly, for example via a spring (e.g., leaf spring). The user-manipulable buttons 240a, 240b may be elongated, extending along a majority or even almost all of the length of the delivery apparatus 100. The use of a pair of user-manipulable buttons 240a, 240b, opposed to one another across the housing 204, which both need to be concurrently activated (e.g., depressed) to cause the delivery apparatus 100 to start dispensing media, advantageously substantially reduces the chance of the delivery apparatus 100 unintentionally discharging media, for example when jogged while carried in a pocket or in a purse.

Alternatively, the opposed (e.g., left and right) peripheral or side wall 208 may be resiliently deformable under manual pressure asserted by squeezing together. Alternatively, one or more sensors may detect inductance, capacitance or force, for example a pair of sensors located at two positions opposed across a longitudinal axis of the housing 204, for instance located along opposed (e.g., left and right) peripheral or side wall 208 applied, to detect a change of inductance or capacitance associated with human skin tissue or a force applied by the closing of a hand.

The use of a pair of user-manipulable buttons 240a, 240b, opposed to one another across the housing 204, which both need to be manipulated or activated (e.g., concurrently depressed) to cause the delivery apparatus 100 to start dispensing media, advantageously substantially reduces the chance of the delivery apparatus 100 unintentionally discharging media, for example when jogged while carried in a pocket or in a purse.

In use, manipulation of the pair of user-manipulable buttons 240a, 240b causes a state of one or more switches 251 (e.g., pair of momentary contact switches) to change (e.g., from an off State to an ON state). The state may be responsive to, for example, a position, a movement or deflection, an amount of applied force or pressure, a measurement of capacitance, a measure of inductance, and/or a measure of resistance. A processor or controller or other circuitry may monitor or otherwise receive the states of the switch(es). In some implementations, the processor or controller or other circuitry can activate the nebulizer 220 in response to detecting or determining that the state of the switch or switches 251 are indicative of a signal to start dispensing media. In some implementations, the delivery apparatus 100 may include one or more sensors (e.g., inertial sensors, one-, two-, three-axis accelerometers, magnetic field sensors, proximity sensors, imagers for facial sensing), and the processor or controller or other circuitry may confirm an orientation of the delivery apparatus 100 is suitable before dispensing media. Thus, in some implementations, media dispensing may only occur when both of two conditions are established: 1) an appropriate user input (e.g., two user-manipulable buttons 240a, 240b concurrently depressed, simultaneously depressed, depressed within 0.5 seconds of one another; quick (e.g., less than 0.5 seconds, less than 0.25 seconds) successive depressions of the two user-manipulable buttons 240a, 240b); and 2) the delivery apparatus 100 is oriented (e.g., upright, upward facing, non-horizontal, outlet port not pointed downward, pointed toward a face) suitably with respect to some reference (e.g., the ground, a face).

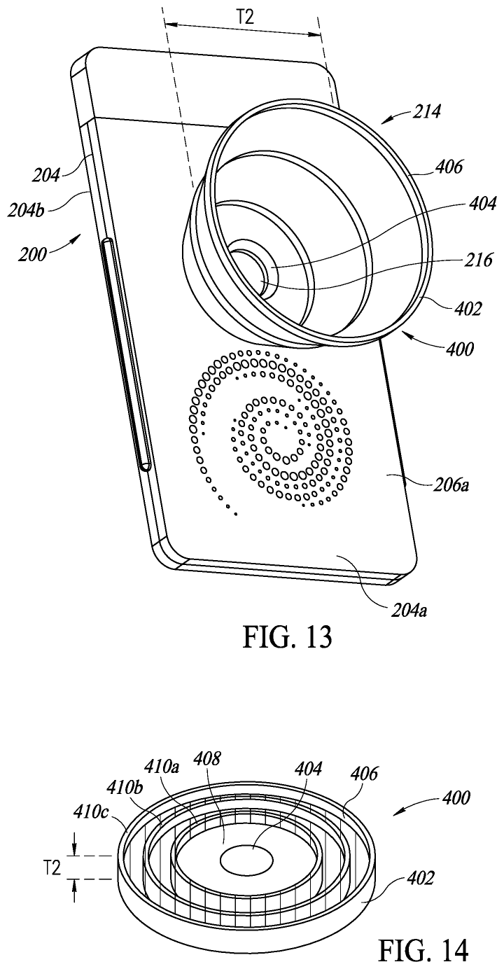

Referring to FIGS. 13 to 16, the delivery apparatus 200 may include a scent concentrator 400 that directs and/or concentrates the media as it is expelled from the delivery apparatus 200 into the external environment 214. Although described herein with reference to the delivery apparatus 200, it will be appreciated that the scent concentrator 400 may be included with the delivery apparatus 100, or any other embodiment of the delivery apparatus as described herein.

The scent concentrator 400 may be supported by the housing 204, for example by one of the pieces 204a, 204b. As shown in the illustrated embodiment, the scent concentrator 400 may be supported by, for example directly coupled to, one of the pair of major walls or faces 206a, 206b. According to one implementation, the scent concentrator 400 may be positioned such that a portion of the scent concentrator 400 at least partially encloses, for example encircles, the outlet port 216.

The scent concentrator 400 may be permanently coupled to the housing 204 or integral therewith, for example such that the scent concentrator 400 cannot be removed from the housing 204 without plastic deformation of at least one of the scent concentrator 400 and the housing 204. According to one implementation, the scent concentrator 400 may be monolithic with the housing 402. The scent concentrator 400 may be removably coupled to the housing 204 such that the scent concentrator 400 may be removed from the housing 204 without plastic deformation of either the scent concentrator 400 or the housing 204. For example, the scent concentrator 400 may be adhesively coupled to the housing 204. According to another example, the scent concentrator 400 and the housing 204 may have corresponding structures, such as a projection and matching recess, which releasably couples the scent concentrator 400 and the housing 204, or some other coupler or mount (e.g., female threaded receptacle or receiver and complimentary male threaded coupler, bayonet mount, detent, retainers and complimentary ears or lugs).

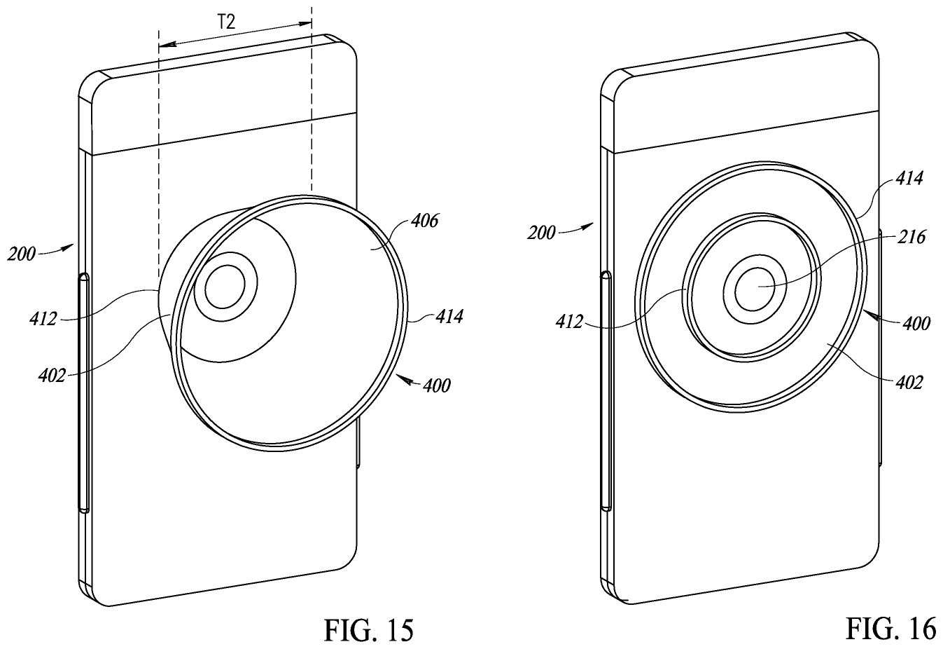

The scent concentrator 400 may include a collapsible body 402 such that the scent concentrator 400 may be transitioned between an expanded configuration (as shown in FIGS. 13 and 15) and a collapsed configuration (as shown in FIGS. 14 and 16). The collapsible body 402 may include a material that is impermeable, such that the media exiting the outlet port 216 cannot pass through the material. According to one implementation, in the expanded configuration the collapsible body has a frustoconical shape.

The collapsible body 402 has a thickness T2 measured from a proximal opening 404, which is proximate the outlet port 216, to a distal opening 406 which may be opposite to the proximal opening 404 with respect to a direction normal to the major wall or face 206a. The distal opening 406 may be sized so as to direct flow of the medium toward a user's face. According to one implementation, the distal opening 406 may be sized so as to allow at least a portion of a user's face, such as a user's nose and/or mouth, to be positioned proximate to or within the distal opening 406.

The distal opening 406, according to one embodiment, may be shaped so as to at least partially enclose a user's nose within a periphery formed by the distal opening 406, while the user's mouth remains outside the periphery formed by the distal opening 406. The portion of the collapsible body 402 that forms the distal opening 406 may include a material, for example a flexible material, that conforms to match a shape of a surface against which the collapsible body 402 is pressed, such that the distal opening forms a seal with the portion of the user's face when pressed against the user's face. Thus, the collapsible body 402 may be shaped so as to form a closed space around a user's nose, while the user's mouth remains outside the closed space. Alternatively, the collapsible body 402 may be shaped so as to form a closed space around both a user's nose and mouth simultaneously.

The collapsible body 402 may include a base surface 408 which faces the housing 204, and forms the proximal opening 404. According to one implementation, the thickness T2 is the maximum dimension of the scent concentrator 400 measured along a direction normal to the surface, for example the major wall or face 206a, to which the scent concentrator 400 is directly coupled.

The thickness T2 is variable such that in the expanded configuration the thickness T2 is greater than when the scent concentrator 400 is in the collapsed configuration. The scent concentrator 400 may have one or more intermediate configurations in which the thickness T2 is greater than when the scent concentrator 400 is in the collapsed configuration and smaller than when the scent concentrator 400 is in the expanded configuration.

As shown in FIGS. 13 and 14, the collapsible body 402 may include a semi-rigid material that is arranged so as to expand and collapse telescopically. When in the collapsed configuration, as shown in FIG. 14, a series of concentric, nested, portions 410a, 410b, 410c, for example rings, are arranged radially about the outlet port 216. The collapsible body 402 may include portions of rigid material, for example portions that include the series of concentric portions 410a, 410b, 410c.

According to one implementation, each of the concentric portions 410a, 410b, 410c may have the same thickness, and that thickness is the thickness T2. According to one implementation, each of the concentric portions 410a, 410b, 410c may have different thicknesses, and the greatest thickness of the concentric portions 410a, 410b, 410c is the thickness T2. The collapsible body 402 may create a hermetic seal between the proximal opening 404 and the distal opening 406.

As shown in FIGS. 15 and 16, the collapsible body 402 may include a semi-rigid material that is arranged so as to expand and collapse non-telescopically. According to one implementation, the collapsible body 402 includes a single membrane that extends from the proximal opening 404 to the distal opening 406, such that the scent concentrator 400 is hermetically sealed between the proximal opening 404 and the distal opening 406. When in the collapsed configuration, as shown in FIG. 16, a proximal frame 412, which forms the proximal opening 404, and a distal frame 414, which forms the distal opening 406 are arranged radially about the outlet port 216.

The housing 204 may include a retention mechanism, for example a recess, a magnet, etc., that releasably receives the distal frame 414 when the scent concentrator 400 is in the collapsed configuration. According to one implementation, each of the proximal frame 412 and the distal frame 414 may have the same thickness, and that thickness is the thickness T2. The collapsible body 402 may include ribs or rings between the proximal opening 404 and the distal opening 406 to support the collapsible body 402 in the expanded configuration.

According to one implementation, the thickness T2 of the scent concentrator 400 in the collapsed configuration is about 25% or less than the thickness T2 of the scent concentrator 400 in the expanded configuration. According to one implementation, the thickness T2 of the scent concentrator 400 in the collapsed configuration is about 10% or less than the thickness T2 of the scent concentrator 400 in the expanded configuration.

U.S. patent application 62/833,366, filed Apr. 4, 2019; U.S. patent application 62/652,069, filed Apr. 3, 2018; U.S. patent application 62/628,395, filed Feb. 9, 2018; U.S. patent application 62/556,974, filed Sep. 11, 2017; U.S. patent application 62/687,970, filed Jun. 21, 2018; and International patent application PCT/US2018/050250, filed Sep. 10, 2018; U.S. patent application 62/908,239, filed Sep. 30, 2019; and U.S. patent application 62/984,697, filed Mar. 3, 2020, are hereby incorporated by reference, in their entireties. The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A delivery apparatus, the delivery apparatus comprising:
a housing having a pair of major faces, the major faces opposed to one another across a thickness of the housing, the housing having an interior between the major faces, a media cartridge receiver sized to receive at least a portion of a media cartridge thereby, and at least one port that provides fluid communications between at least a portion of the interior of the housing and an external environment external to an exterior of the housing;
a nebulizer housed in the housing, the nebulizer including a vibration member and an actuator coupled to cause the vibration member to vibrate at one or more frequencies;
a pair of contacts manually engageable by a user from the exterior of the housing;
a drive circuit comprising at least one switch responsive to manual engagement of the contacts, the drive circuit coupled to drive the actuator in response an occurrence of at least one condition, the at least one condition including at least a manual engagement of each of the pair of contacts at least within a defined time threshold of one another.

2-12. (canceled)

13. The delivery apparatus of claim 1 wherein the drive circuit comprises at least one sensor response to an orientation of the delivery apparatus.

14. The delivery apparatus of claim 13 wherein the at least one sensor responsive to an orientation of the delivery apparatus comprises at least one of an accelerometer or a proximity sensor.

15. The delivery apparatus of claim 13 wherein the at least one condition includes the orientation of the delivery apparatus being within a defined range of orientations at least within a threshold period of the manual engagement of each of the pair of contacts at least within the defined time threshold of one another.

16. The delivery apparatus of claim 1 wherein the at least one condition includes at least two successive manual engagements of each of the pair of contacts at least within a defined successive engagement time threshold.

17. The delivery apparatus of claim 1 wherein the media cartridge receiver comprises a slot in the housing and a gasket, the gasket having an opening sized and shaped to closely receive a neck of the media cartridge.

18. The delivery apparatus of claim 1 wherein a front one of the major faces of the housing is planar, and a rear one of the major faces of the housing is non-planar.

19. The delivery apparatus of claim 1 wherein the media cartridge receiver sized to closely, removably, receive at least a portion of the media cartridge thereby.

20. The delivery apparatus of claim 1 wherein at least an inner portion of a fluid path in the housing has a hydrophilic surface or a hydrophilic coating or film.

21. The delivery apparatus of claim 1 wherein at least an inner portion of a fluid path in the housing has a rough surface to increase a turbulence in a fluid flow.

22. The delivery apparatus of claim 1, further comprising a scent concentrator supported by the housing, the scent concentrator including a body having a proximal opening that at least partially encircles the at least one port, the collapsible body further having a distal opening and a maximum thickness, the maximum thickness measured from the proximal opening to the distal opening, and the body is collapsible and expandable such that the maximum thickness is variable.

23. The delivery apparatus of claim 22 wherein the scent concentrator is transitionable from a collapsed configuration to an expanded configuration, in the collapsed configuration the maximum thickness is less than 25% of the maximum thickness when the scent concentrator is in the expanded configuration.

24. The delivery apparatus of claim 23 wherein the maximum thickness in the collapsed configuration is less than 10% of the maximum thickness when the scent concentrator is in the expanded configuration.

25. The delivery apparatus of claim 22 wherein the scent concentrator is transitionable from a collapsed configuration to an expanded configuration, and in the collapsed configuration the body forms a plurality of concentric shapes.

26. The delivery apparatus of claim 25 wherein a center of the plurality of concentric shapes is positioned within the at least one port.

27. The delivery apparatus of claim 25 wherein the plurality of concentric shapes include a plurality of concentric rings.

28-71. (canceled)
